(19) United States
(12) Patent Application Publication
(10) Pub. No.: US 2011/0008772 A1
(43) Pub. Date: Jan. 13, 2011

(54) PROBES FOR DETECTING MYCOBACTERIUM TUBERCULOSIS AND MYCOBACTERIUM TUBERCULOSIS COMPLEX AND METHOD USING THE SAME

(75) Inventors: Hsin-Chih Lai, Taipei City (TW); Jann-Yuan Wang, Taipei City (TW); Po-Ren Hsueh, Taipei City (TW); Pi-Chi Soo, Taipei City (TW); Yu-Tze Horng, Taipei City (TW); Kai-Chih Chang, Taipei City (TW); Chia-Chen Lu, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: INSTITUTE FOR BIOTECHNOLOGY AND MEDICINE INDUSTRY, Taipei City (TW)

(21) Appl. No.: 12/644,963

(22) Filed: Dec. 22, 2009

(30) Foreign Application Priority Data
Jul. 7, 2009 (TW) 098122884

(51) Int. Cl. C12Q 1/68; C07H 21/00

(52) U.S. Cl. 435/6; 536/24.32

(57) ABSTRACT

The present invention relates to a probe for detecting Mycobacterium tuberculosis complex (MTBC) and Mycobacterium tuberculosis (MTB) from clinical specimens. The method mainly comprised two steps: (1) amplifying a target DNA of the clinical specimens through nested PCR; and (2) hybridizing to the target DNA with a gold nanoparticle probe. The presence of MTB or MTBC was detected after the absorbance at 525 nm was determined with a spectrophotometer during hybridization at a defined time. Direct observation on color changes can also be detected after a longer incubating time. The merits of this assay include being easy to operate, sophisticated detection equipment is not necessary, low cost, time saving, and, high sensitivity and specificity.

### BACKGROUND OF THE INVENTION

1. Field of the Invention

The present invention relates to a probe for bacterial identification, in particular to a probe for detecting Mycobacterium tuberculosis and Mycobacterium tuberculosis complex.

2. The Prior Arts

Tuberculosis (TB) is a common lethal disease in the developing countries, which is responsible for approximately two million deaths annually. Recently the global number of TB cases is rising at a rate of 2% per year according to World Health Organization tuberculosis fact sheet. Members of Mycobacterium tuberculosis complex (MTBC) are the causative agents of tuberculosis in humans, including M. tuberculosis, M. africanum, M. microti and M. canetti. Mycobacterium tuberculosis is the principal etiologic agent of tuberculosis in humans. The primary site of infection is in the lungs.

Conventionally, smear microscopy and culture methods are widely used in diagnosis of TB. However, the former is insensitive and the latter takes up to 6-8 weeks to provide a result, limiting the value of these methods in aiding diagnosis and immediate decisions on treatment. Many nucleic acid amplification based detection systems have also been developed as rapid tests for the direct identification of Mycobacterium tuberculosis complex (MTBC) from clinical specimens. MTBC members differ widely in terms of host tropisms, phenotypes, and pathogenicity though the mycobacteria grouped in the MTBC are closely related based on DNA-DNA hybridization, multilocus enzyme electrophoresis and 16S rDNA nucleotide acid sequence identity level. It is intriguing that some species are either exclusively human (M. tuberculosis, Mycobacterium africanum, and Mycobacterium canetti) or rodent (Mycobacterium microti) pathogens. Others either have a wide host spectrum (Mycobacterium bovis) or are used as a vaccine strain (M. bovis BCG). Differentiation of M. tuberculosis (MTB) from the other members of the Mycobacterium tuberculosis complex (MTBC) is thus important both for the treatment of patients as well as for epidemiological study, in particular to areas of the world where tuberculosis has reached epidemic proportions, the transmission of M. bovis between animals or animal products and humans is a particular problem, or where the vaccine strain M. bovis BCG causes pediatric infections.

Sequence-specific methods for detecting polynucleotides are critical to the diagnosis of pathogenic diseases. Most detection systems use the hybridization of the target polynucleotide with oligo- or poly-nucleotide probes containing covalently linked reporter groups. Many commercial identification systems are available at present. However, the high costs and long detection time are a constraint on their use, especially in developing countries.

### SUMMARY OF THE INVENTION

The conventional methods for detection of Mycobacterium tuberculosis (MTB) and Mycobacterium tuberculosis complex (MTBC) have the problems of complicated operation, time consuming and high cost as mentioned in the prior arts.

Due to the limitation of detection in MTB and MTBC as described above, the primary objective of the present invention is to provide a probe for detecting of MTB and MTBC in a specimen, wherein the probe is selected from the group consisting of nucleotide sequences of SEQ ID NO: 1 to 4 and the completely complementary sequences thereof, and the probes are labeled with a reporter. Furthermore, a first probe set used for MTBC detection is SEQ ID NO: 1/SEQ ID NO: 2 or the complementary nucleotide sequences thereof, and the nucleotide sequences are labeled with reporters; the a second probe set used for MTBC detection is SEQ ID NO: 3/SEQ ID NO: 4 or the complementary nucleotide sequences thereof, and the nucleotide sequences are labeled with reporters. The specimens are sputum or respiratory secretions, and the reporter is selected from the group consisting of biotin, digoxigenin, fluorescence materials, nano-gold particles, nano-silver particles, nano-palladium (Pd), nano-ruthenium (Ru), nano-platinum (Pt), and any other conjugating color indicators.

Another objective of the present invention is to provide a method for identification a fragment of IS6110 gene of Mycobacterium tuberculosis complex (MTBC) and Rv3618 gene of Mycobacterium tuberculosis (MTB).

To overcome the drawbacks of the known technology and fulfill the objectives of the present invention, a probe labeled with a reporter was employed to detect MTB and MTBC in a specimen, wherein the reporter was a gold nanoparticle. The method for the identification mainly comprised two steps: (1) amplifying a target DNA from the specimen through PCR; and (2) hybridizing to the target DNA with a gold nanoparticle probe. The presence of Mycobacterium tuberculosis (MTB) or Mycobacterium tuberculosis complex (MTBC) was detected after the absorbance at 525 nm was determined with a spectrophotometer during hybridization at a defined time. Direct observation on color changes can also be detected after a longer incubating time. Nested PCR can be used for DNA amplification on the specimen.

Nested PCR combined with the addition of gold nanoparticle probe for hybridization in the present invention is a preferred way in direct and rapid diagnosis of Mycobacterium tuberculosis complex (MTBC) and Mycobacterium tuberculosis (MTB) from clinical sputum samples. The merits of the present invention include being easy to operate, sophisticated detection equipment is not necessary, low cost, time saving, and, high sensitivity and specificity. Similar to other molecular detection systems, the sensitivity of the present invention is dependent on whether there is enough DNA to be used. The remaining detection steps of the present invention are simple and rapid as long as enough target DNA is available. Meanwhile, the method of the invention can also be applied as a plateform in DNA diagnosis other than MTBC and MTB detection. Compared with conventional DNA detection systems whose specificity is mainly determined by the design of DNA primer sequences, the addition of specific gold nanoparticle probe of the present invention further improves the specificity by reducing the background noise of non-specific DNA. The probes of the present invention showed a sensitivity of 96.6% and specificity of 98.9% toward Mycobacterium tuberculosis complex (MTBC) detection, and a sensitivity of 94.7% and specificity of 99.6% toward Mycobacterium tuberculosis (MTB) detection were shown. In addition, the reading of the results can be conveniently achieved by direct observation besides spectrophotometric analysis if incuting time is long enough. This method can be developed for automation analysis with spectrophotometric detection in large scale DNA diagnosis.

### BRIEF DESCRIPTION OF THE DRAWINGS

FIG. 1 shows the schematic illustration of hybridization of gold nanoparticle probes and target DNA and the color and absorbance changes after hybridization.

FIG. 2 shows the hybridization results with different dilution of DNA.

FIG. 3 shows the detection results of gold nanoparticle probes assay toward different bacterial strains for detection of IS6110 and Rv3618.

FIG. 4 shows the confirmation of hybridization by heating and cooling of the gold nanoparticle probe and DNA mixture.

### DETAILED DESCRIPTION OF THE PREFERRED EMBODIMENT

These and other features, advantages, and benefits of the present invention can be understood by one of ordinary skill in the arts upon careful consideration of the detailed description of representative embodiments of the invention in connection with the accompanying drawings.

The method for the identification in the present invention mainly comprised of two steps: (1) amplifying a target DNA of the specimen through nested PCR; and (2) hybridizing to the target DNA with a gold nanoparticle probe. The preparation processes before detection include specimen collection and processing, gold nanoparticles preparation, preparation of oligonucleotide/Au nanoparticle conjugates and the like.

### Example 1

### 1. Specimen Collection and Processing

A total of 600 sequential clinical sputum specimens were collected from the Mycobacteriology Laboratory, Department of Laboratory Medicine, National Taiwan University Hospital. An equal volume of NaOH-citrate-N-acetyl-L-cysteine solution was added to the sputum sample at room temperature for 15 min After centrifugation, the precipitate was resuspended in 1 ml of phosphate-buffered saline (pH 7.4).

### 2. Culture and Biochemical Methods for Diagnosis of Mycobacterium tuberculosis Complex (MTBC) and Mycobacterium tuberculosis (MTB)

The Lowenstein-Jensen (LJ) slants (Difco, USA) and Middlebrook 7H11 medium plates (Becton-Dickinson, USA) were inoculated with 250 μl of decontaminated sample suspension, incubated at 37° C. with 5% CO₂. An inverted light microscope was used to observe mycobacterial growth during weeks 2-8 after inoculation. The guidelines of the US Center for Disease Control and Prevention were followed for the determination of positive mycobacterial growth. The indication test tubes of BACTEC MGIT 960 system (Becton-Dickinson Diagnosis Instrument System, USA) were used for prevention of mycobacterial over growth. Identification of bacterial strains as Mycobacterium tuberculosis complex (MTBC) and/or Mycobacterium tuberculosis (MTB) is mainly based on the routine morphological and biochemical assays. Cells were further confirmed to species level by 16S rDNA sequence analysis if ambiguous identification results were obtained by culture methods.

### 3. Mycobacterium ATCC Reference Strains

A total of 23 Mycobacterium reference strains were obtained from ATCC (American Type Culture Collection, Manassas, Va.) as controls in the invention. The examples include, but are not limited to, the strains listed in table 1: M. tuberculosis H37Rv (ATCC 27294), M. bovis (ATCC 19210), M. microti (ATCC 19422), M. avium-intracellulare complex (ATCC 35761), M. kansaii (ATCC 12478), M. marinum (ATCC 927), M. chelonae (ATCC 35752), M. abscessus (ATCC 19977), M. fortuitum (ATCC 6841), M. smegmatis (ATCC 35798), M. xenopis (ATCC 19250), M. asiaticum, M. haemophilum, M. mucogenicum, M. malmoense, M. terrae, M. triviae, M. vaccae, M. flavescence, M. gastri, M. gordonae, M. scrofulaceum and M. simiae.

TABLE 1

| | Gold nanoparticle | |
|---|---|---|
| | probes assay | |
| Reference strains* | IS6110 | Rv3618 |
| Mycobacterium spp. | | |
| M. tuberculosis H37Rv (ATCC 27294) | + | + |
| M. bovis (ATCC 19210) | + | − |
| M. microti (ATCC 19422) | + | − |
| M. avium-intracellulare complex (ATCC 35761) | − | − |
| M. kansaii (ATCC 12478) | − | − |
| M. marinum (ATCC 927) | − | − |
| M. chelonae (ATCC 35752) | − | − |
| M. abscessus (ATCC 19977) | − | − |
| M. fortuitum (ATCC 6841) | − | − |
| M. smegmatis (ATCC 35798) | − | − |
| M. xenopis (ATCC 19250) | − | − |
| M. asiaticum | − | − |
| M. haemophilum | − | − |
| M. mucogenicum | − | − |
| M. malmoense | − | − |
| M. terrae | − | − |
| M. triviae | − | − |
| M. vaccae | − | − |
| M. flavescence | − | − |
| M. gastri | − | − |
| M. gordonae | − | − |
| M. scrofulaceum | − | − |
| M. simiae | − | − |

*Reference strains were either ATCC strains or clinically isolated strains identified by biochemical assays and 16S rDNA sequencing.
+: aggregation;
−: no aggregation.

### 4. Preparation of Mycobacterial DNA from Sputum Specimens and Nested PCR

Decontaminated sample suspensions (100-300 μl) were mixed with equal volume of wash buffer (Tris-HCl buffer, pH 8). After vortexing for 20 s, samples were subject to centrifugation at 13,000 rpm for 10 min before discarding the supernatant. The precipitated pellet was resuspended and lysed in lysis buffer (KOH, pH 13.1) at 95° C. for 15 min before being neutralized by neutralization buffer (HCl and acetic acid, pH 1.2). To evaluate the sensitivity of this assay, the chromosomal DNA of M. tuberculosis H37Rv (ATCC 27294) grown on Middlebrook 7H11 agar plates was extracted with the same procedures, and were serially diluted in normal saline before PCR. A 5 μl of crude extract suspension was then transferred to an eppendorf tube containing 50 μl of amplification reagent (10 mM Tris-HCl pH 8.3, 50 mM KCl, 1.5 mM MgCl₂, 0.2 mM dNTP, 20 μmol for each primer and 2.5 units Taq DNA polymerase (Takara, Japan)) for nested PCR with primers listed in Table 2. The nested PCR carried out in a thermal reactor (Biometra, Germany) included the following procedures:

For first PCR reaction:
(1) incubating at 94° C. for 5 min;
(2) amplifying long DNA fragments (245 by and 326 by for IS6110 and Rv3618) respectively with primer pairs of INS1-INS2 (SEQ ID NO. 5 and SEQ ID NO. 6) and Rv3618F-Rv3618R (SEQ ID NO. 9 and SEQ ID NO. 10) since IS6110 and Rv3618 are unique DNA fragments to Mycobacterium tuberculosis complex (MTBC) and Mycobacterium tuberculosis (MTB), which can therefore be used in diagnostic of MTBC and MTB; the PCR parameters are as follow:

A. 94° C. for 30 s;

B. 64° C. for 15 s;

C. 72° C. for 30 s;

D. Repeat A-C for 40 cycles.
(3) incubating at 72° C. for 1 min; the PCR mixtures without template DNA were used as negative controls.

5 μl of PCR mixture from first PCR reaction was used as a template for the second PCR reaction (10 mM Tris-HCl pH 8.3, 50 mM KCl, 1.5 mM MgCl₂, 0.2 mM dNTP, 20 μmol for each primer and 2.5 units Taq DNA polymerase (Takara, Japan). A total of 14 cycles were performed to amplify the 110 by (for IS6110) and 124 by (for Rv3618) fragments with 8 primers: INS1, INS2, G-IS6110F, G-IS6110R, Rv3618F, Rv3618R, G-3618F, G-3618R as listed in Table 2 (corresponding to SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12).

### 5. Clinical Evaluation for Tuberculosis Patients

All the medical records, including the history, symptoms, signs, results from radiology, pathology and microbiology and the follow-ups were recorded as described (Wang et al. 2004).

### Example 2

### 1. Au Nanoparticles Preparation

Au colloids were prepared by the Natan method (Freeman et al. 1995). Briefly, 39.37 mg of HAuCl₄.3H₂O was dissolved in 100 ml of distilled, deionized water by heating and vigorously stirring. Then 10 ml of 38.8 mM sodium citrate solution was added as the solution was boiling. Finally the tetrachloroauric solution turned claret, with nanoparticle concentration of approximately 20 nM, and with the mean diameter of Au at 13.7±0.8 nm

### 2. Preparation of Oligonucleotide/Au Nanoparticle Conjugates

Au nanoparticles capped with 3′ and 5′-thiol terminated oligonucleotides were prepared following Mirkin's strategy (Storhoff et al. 1998). The synthesized gold nanoparticles were centrifuged for 7 min at 10,000 rpm to remove the excess citrate and brought into 10 mM phosphate buffer (pH 7). The Au nanoparticles were derivatized by using 1 ml of a colloidal solution with 50D₂₆₀ (165 μg) of oligonucleotides (5′-HS-A 10-oligomer-3′ or 5′-oligomer-A10-5H-3′) to age for 16 h. The sequences of oligonucleotides SEQ ID NO: 1, SEQ ID NO: 2, SEQ ID NO: 3 and SEQ ID NO: 4 were listed in Table 2. The solution was then transferred to 0.1M NaCl, 10 mM phosphate buffer (pH 7), and allowed to stand for 42 h. The resulting aged solution was centrifuged for 20 min at 13,000 rpm twice to attain red precipitates. The resulting precipitates were then washed with 1 ml of a 0.1 M NaCl, 10 mM phosphate buffer (pH 7) solution and then re-suspended in 1 ml of 0.1 M NaCl, 10 mM phosphate buffer (pH 7). All procedures were carried out at room temperature (25° C.).

TABLE 2

| | Application |
|---|---|
| Primer sequence (5′→3′) | characteristics |
| INS1: CGTGAGGGCATCGAGGTGGC | 1^{st} PCR for amplifi- |
| INS2: GCGTAGGCGTCGGTGACAAA | cation of IS6110 |
| G-IS6110F: CTCGTCCAGCGCCGCTTCGG | 2^{nd} PCR for amplifi- |
| G-IS6110R: GCGTCGGTGACAAAGGCCAC | cation of IS6110 |
| Rv3618F: ATTGCACATCCGCCCC | 1^{st} PCR for amplifi- |
| Rv3618R: GGACAAACCCTGCCGC | cation of Rv3618 |
| G-3618F: CGACTGGTTCACCCTG | 2^{nd} PCR for amplifi- |
| G-3618R: TAACAGCGACGTGCCCAG | cation of Rv3618 |
| Gold nanoparticle | |
| probes sequence | |
| SEQ ID NO: 1: | Hybridization to |
| SH-A10-CACCCATCGTCTGGAGTGG | target DNA for |
| SEQ ID NO: 2: | IS6110 |
| CCAAGCGGATGCACCGG-A10-SH | |
| SEQ ID NO: 3: | Hybridization to |
| SH-A10-AGCCATGATTTCGCCATCG | target DNA for |
| SEQ ID NO: 4: | Rv3618 |
| CTGGGCACGTCGCTGTT-A10-SH | |

Though the present example used gold nanoparticle to label oligonucleotide probes, the types of probe label were not restricted. Those skilled in the arts will understand that the probes can be labeled with a reporter such as biotin, digoxigenin, fluorescence materials, nano-gold particles, nano-silver particles, nano-palladium (Pd), nano-ruthenium (Ru), nano-platinum (Pt), or any other conjugating color indicators.

### Example 3

### Application of Gold Nanoparticle—SEQ ID NO: 1/SEQ ID NO: 2/SEQ ID NO: 3/SEQ ID NO: 4 Conjugated Probes in the Detection of MTBC and MTB

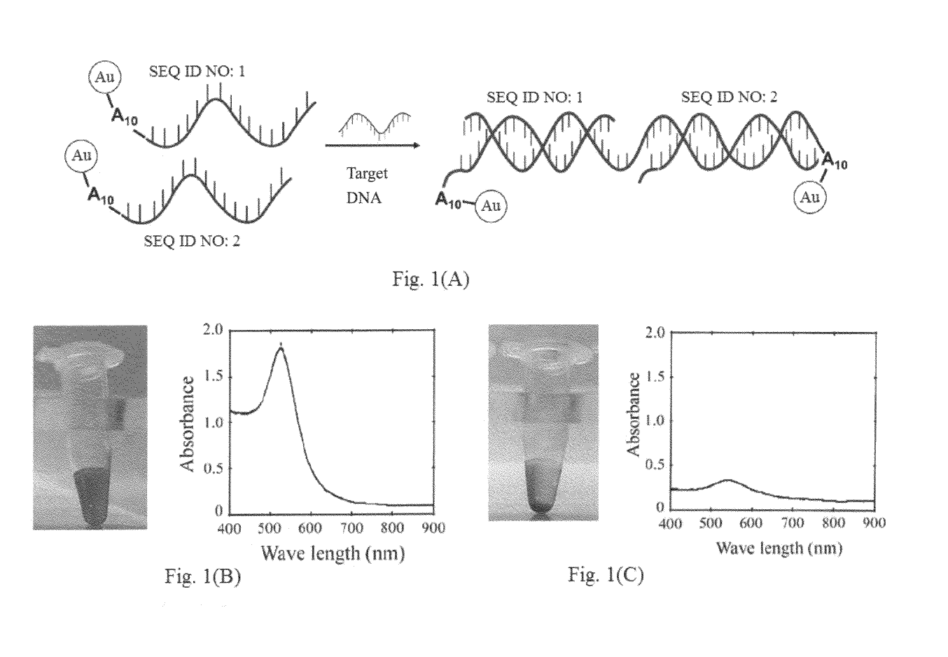

Detection of the presence of target DNA was achieved by the addition of 100 μl of abovementioned probes SEQ ID NO: 1, SEQ ID NO: 2, SEQ ID NO: 3 and SEQ ID NO: 4 respectively at the final concentration of 20 nM. The mixture suspensions were denatured at 95° C. for 10 min, and cooled down to 55° C. for DNA hybridization for 2 h. FIG. 1A shows the schematic illustration of hybridization of probes and target DNA. The color and absorbance changes after hybridization are shown in FIG. 1B and FIG. 1C. The DNA was denatured by heat after the PCR reaction. The gold nanoparticle probes (SEQ ID NO: 1 and SEQ ID NO: 2 as probes, and IS6110 as the DNA to be assayed in this example) were added to the heat denatured PCR products, and incubated at 55° C. for DNA hybridization for 2 h. The absorbance of the solution was measured by spectrophotometer. When singlestranded target DNA was absent in the solution, the color and absorbance pattern at 525 nm were not changed (FIG. 1B). On the contrary, the gold nanoparticle probes aggregated with target DNA through hybridization if specific target DNAs were present in the solution, resulting in decrease in absorbance of the solution at 525 nm and change of color from red to purple (OD₅₂₅ nm) (FIG. 1C). Although color shift could be detected by spectrophotometry as early as 2 h after the addition of probes, a significant difference could also be observed at about 4 h by direct observation. Due to a red shift in the surface plasmon resonance (SPR) of Au nanoparticles, the positive reaction means a change of color from red to purple and the aggregation of gold nanoparticle probes. The cut-off value was 1.698 at OD₅₂₅ nm determined by the average absorbance values minus three standard deviations of 20 control samples without target DNA.

### Example 4

### Detection Sensitivity with IS6110 from Various Concentration of Mycobacterium tuberculosis (MTB)

The detection sensitivity of the present invention is related to the amounts of amplified target DNA through PCR. Either single PCR reaction or nested PCR depended on the DNA amounts in original samples. For example, using DNA extracted from a single colony as the DNA template, a single PCR would be enough for subsequent assay. In comparison, nested PCR or other high amplification efficiency methods are suggested for detecting target DNA from clinical sputum samples.

To evaluate the sensitivity of the present invention, the chromosomal DNA of M. tuberculosis H37Rv (ATCC 27294) grown on Middlebrook 7H11 agar plates was extracted and serially diluted before PCR. Same volumes of sample from different dilution were taken as templates for nested PCR amplification, followed by hybridization to evaluate the sensitivity of the assay.

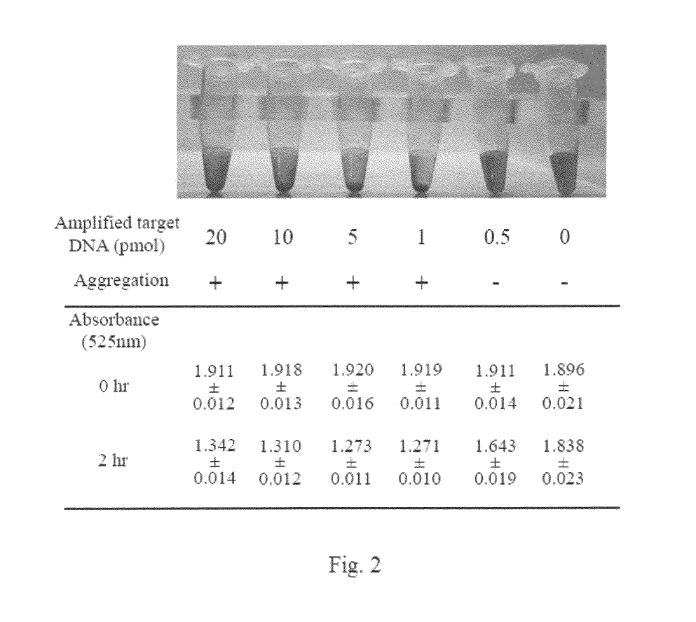

FIG. 2 showed the hybridization results with different dilution of DNA. The detection limit of gold nanoparticle probes assay was determined using amplified IS6110 DNA fragments from M. tuberculosis H37Rv genomic DNA. The amplified DNA was quantified and serially diluted from 20 μmol to 0.5 μmol after PCR amplification. The amplified DNA was denatured at 80° C. for 5 min, and subsequently 20 μM of SEQ ID NO: 1 and SEQ ID NO: 2 gold nanoparticle probes were added, followed by incubation at 55° C. for 2 h or 4 h. The absorbance (OD_{525 nm}) was around 1.913 at 0 h for all samples. At 2 h after probe addition, only the absorbance of negative control sample remained basically unchanged, the absorbances of samples containing 0.5 μmol target DNA were reduced to around 1.643. Absorbances were further reduced to 1.271-1.342 for samples containing 1-20 μmol DNA.

Briefly, the detection at levels as low as 0.5 μmol of the target DNA showed a positive reaction by spectrophotometric analysis 2 h after probe addition. Results with a similar detection limit could also be obtained by direct observation 4 h after probes addition.

### Example 5

### Identification of Strains in Mycobacterium tuberculosis Complex (MTBC)

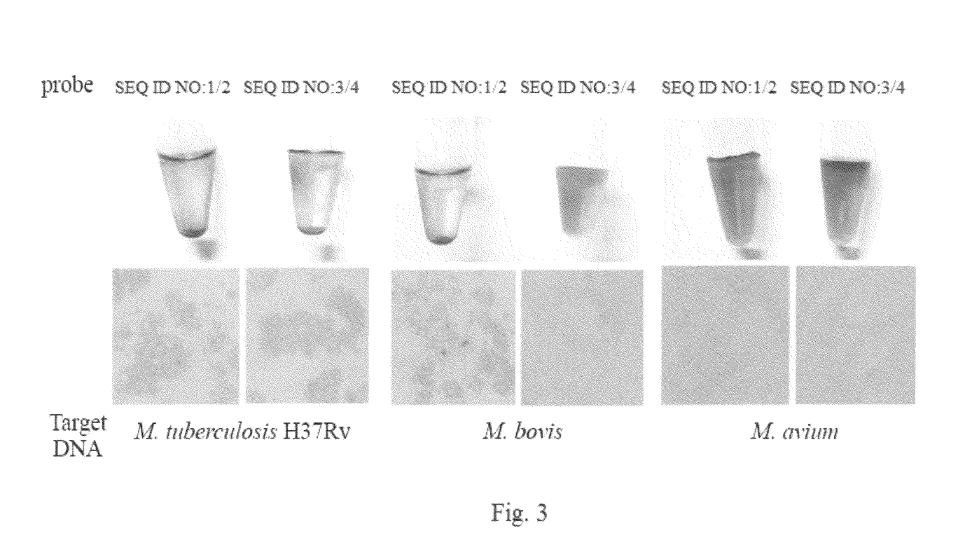

Refers to FIG. 3, the uses of gold nanoparticle probes assay toward different strains for detection of IS6110 and Rv3618. Genomic DNAs prepared from M. tuberculosis H37Rv (ATCC 27294), M. bovis (ATCC19210), and M. avium (ATCC35761) were used as templates for PCR amplification, wherein, M. tuberculosis H37Rv contains both IS6110 and Rv3618 gene while M. bovis contains IS6110 only. DNA fragments amplified with primer pairs G-IS6110F (SEQ ID NO. 7)/G-156110R (SEQ ID NO. 8) and G-3618F (SEQ ID NO. 11)/G-3618R (SEQ ID NO. 12) listed in Table 2 were unique DNAs for Mycobacterium tuberculosis complex (MTBC) and Mycobacterium tuberculosis (MTB). A specific 110 by and a 124 by DNA fragment can be amplified through PCR with templates of IS6110 and Rv3618 genes respectively. These PCR products were used for subsequent gold nanoparticle probes assay.

FIG. 3 showed the detection of target DNA after addition of two pairs of gold nanoparticle probes designed for hybridization with IS6110 and Rv3618 target DNA respectively. Direct observation on color changes of M. tuberculosis H37Rv were shown at 4 h after probes addition, which confirmed the presence of both IS6110 and Rv3618 gene fragments. For M. bovis ATCC19210, a member of MTBC, a positive reaction was only observed with primer pair of SEQ ID NO. 1/SEQ ID NO. 2, which indicated the presence of IS6110. No color change was observed in M. avium ATCC35761 with primer pairs of SEQ ID NO. 1/SEQ ID NO. 2 and SEQ ID NO. 3/SEQ ID NO. 4, which indicated that M. avium is not an Mycobacterium tuberculosis complex (MTBC) member. In concordance with color change, gold nanoparticles aggregation was clearly observed under light microscopy under the 400× light microscopy (lower panel on FIG. 3).

Therefore, the probes of the present invention not only identify Mycobacterium tuberculosis complex (MTBC), but also distinguished Mycobacterium tuberculosis (MTB) from MTBC, and differentiated M. bovis based on these results.

The gold nanoparticle probe was used to identify the 23 Mycobacterium spp. reference strains listed in Table 1 to test the specificity. These Mycobacterium strains grown on Middlebrook 7H11 plates were used for identification. Positive reactions for both IS6110 and Rv3618 probes were obtained in identifying M. tuberculosis H37Rv. In comparison, IS6110-positive, but Rv3618-negative reaction was present in detecting M. bovis ATCC19210 and M. microti ATCC 19422 which are MTBC members. For the non-tuberculous Mycobacterium (NTM) strains, no positive reactions were observed with both probes. Briefly, this assay specifically identified MTBC and further differentiated MTB from MTBC, and no cross-reaction with the other NTM bacterial strains was observed.

### Example 6

### The Color Shift of Gold Nanoparticle Probes in the Present Invention is Caused by the Hybridization with Target DNAs and a Reversible Aggregation

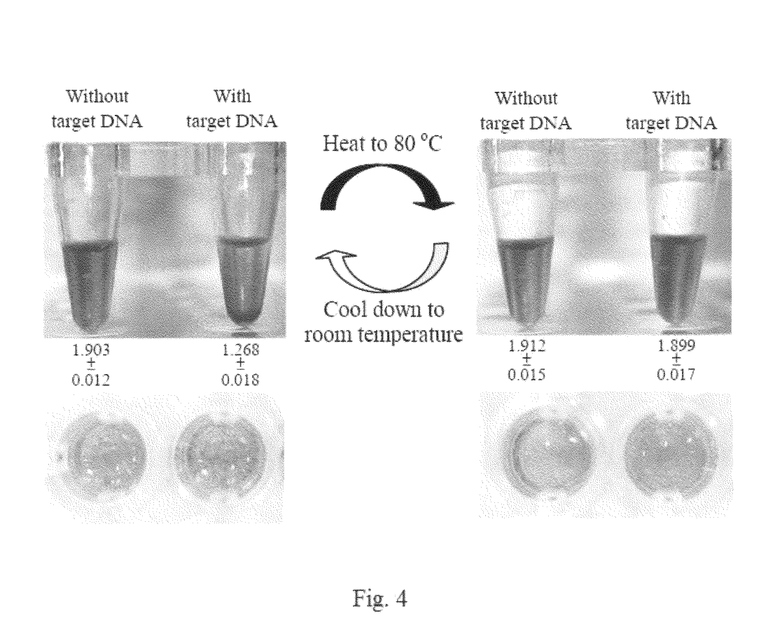

Referring to FIG. 4, hybridization was confirmed after heating and cooling of the gold nanoparticle probe and DNA mixture. To confirm the color change and aggregations are not artificial phenomena, the mixtures were heated to 80° C. to denature the DNAs after positive hybridization. The color of the mixtures turned from purple to red, and the aggregated particles were resuspended again in the solution, as shown by direct observation (right panel in FIG. 4). The absorbances (OD_{525 nm}) became 1.899±0.017 in experimental group with target DNAs. When the mixtures were cooled down again, the same color shift and gold nanoparticles aggregation were observed (lower panel in FIG. 4). The absorbances (OD_{525 nm}) became 1.268±0.018 in experimental group with target DNAs. These results indicated color shift observed in this assay is reversible and is a nature of hybridization between template and probe DNAs.

### Example 7

### Comparisons of Sensitivity and Specificity of Mycobacterium tuberculosis Complex (MTBC) Detection Between Gold Nanoparticle Probe Assay in the Present Invention and Conventional Diagnostic Methods

A total of 600 consecutive clinical sputum specimens subjected to routine mycobacteria identification in National Taiwan University Hospital were analysed with the assay of the present invention. Purified genomic DNAs as templates and primer pairs of INS1 (SEQ ID NO. 5)/INS2 (SEQ ID NO. 6) and Rv3618F (SEQ ID NO. 9)/Rv3618R (SEQ ID NO. 10) (Table 2) were subjected to PCR to generate the 1st DNA fragments of MTBC and MTB, respectively. They were used in amplification of the 2nd DNA fragments using the primer pairs G-IS6110F (SEQ ID NO. 7)/G-156110R (SEQ ID NO. 8) and G-3618F (SEQ ID NO. 11)/G-3618R (SEQ ID NO. 12) (Table 2). After DNA amplification, probe hybridization results were determined by spectrophotometer and compared with those obtained from conventional culture methods (Table 3).

TABLE 3

| | |
|---|---|
| Gold nanoparticle probes | |
| (IS6110) assays | Culture* |

| (no. of samples) | Positive | Negative |
|---|---|---|
| Positive (62) | 56 | 6 |
| Negative (538) | 2 | 536 |
| Total (600) | 58 | 542 |

*Culture and biochemical diagnosis results were from mycobacteriology laboratory, National Taiwan University Hospital (NTUH). Sensitivity: 96.6%, Specificity: 98.9%; positive predictive value: 90.3%, negative predictive value: 99.6%. The results were read 2 h after probes addition by spectrophotometry at OD_{525 nm}.

Referring to Table 3, a total of 58 specimens were identified to contain Mycobacterium tuberculosis complex (MTBC) organisms among the 600 specimens identified by traditional culture and biochemical methods. In comparison, 2 out of 58 specimens were detected negative with gold nanoparticle probe assay. PCR using re-extracted genomic DNAs was repeated with gold nanoparticle probe assay. These 2 strains were confirmed to be MTBC, IS6110 positive. Therefore, the results might be caused by low genomic DNA extracted or some PCR inhibitors in the sputum specimens.

At the same time, a total of 62 specimens, which were identified to be IS6110 positive with gold nanoparticle probe assay. In comparison, 6 out of 62 specimens were negative in traditional culture results. Further clinical assessment showed that 2 out of the 6 patients showed significant clinical syndromes of MTBC infection. In summary, a sensitivity of 96.6% and specificity of 98.9% were shown in MTBC detection using the gold nanoparticle probe assay.

### Example 8

### Comparisons of Sensitivity and Specificity of Mycobacterium tuberculosis (MTB) Detection Between Gold Nanoparticle Probe Assay in this Invention and Conventional Diagnostic Methods

Referring to table 4, the gold nanoparticle probe assay was compared with those obtained from conventional culture methods.

TABLE 4

| | |
|---|---|
| Gold nanoparticle probes | |
| (Rv3618) assays | Culture** |

| (no. of samples) | Positive | Negative |
|---|---|---|
| Positive (56) | 54 | 2 |
| Negative (544) | 3 | 541 |
| Total (600) | 58 | 543 |

**Culture and biochemical diagnosis results were from mycobacteriology laboratory, National Taiwan University Hospital (NTUH). Sensitivity: 94.7%, Specificity: 99.6%; positive predictive value: 96.4%, negative predictive value: 99.2%. The results were read 2 h after probes addition by spectrophotometry at OD_{525 nm}.

A total of 58 specimens were identified to contain MTBC among the 600 specimens identified by traditional culture and biochemical methods (Table 3). Among the 58 MTBC culture-positive clinical specimens, 57 specimens were culture confirmed to contain MTB, and one specimen with M. bovis (Table 4). The specimen which contained M. bovis was also detected Rv3618 (MTB) negative with gold nanoparticle probe assay. As shown in Table 4, a total of 54 (54/57) specimens were detected Rv3618 positive by gold nanoparticle probe assay. Thus 3 specimens were culture-positive for MTB but were Rv3618-negative with the gold nanoparticle probe assay. Detailed analysis of these 3 specimens indicated 2 were IS6110-negative and one was IS6110-positive. These bacterial cells postulated to be deficient of Rv3618 in RD9 region. PCR using a designed RD9-flanking primers followed by agarose gel electrophoresis was performed with DNA templates prepared from this clinical isolate, M. tuberculosis H37Rv, and M. bovis ATCC19210. RD9 region was shown to be absent from this clinical isolate and M. bovis ATCC19210, while present in M. tuberculosis H37Rv (data not shown). It's indicated that RD9 region might be absent in some of the mycobacterial spp.

At the same time, 2 samples showed Rv3618-negative in conventional method but Rv3618-positive in gold nanoparticle probe assay. Further analysis showed that these 2 samples were also IS6110 positive, indicating MTB organisms were indeed contained in these two samples. In summary, a sensitivity of 94.7% and specificity of 99.6% were shown in MTB detection using the gold nanoparticle probe assay.

In conclusion, the detection assay using the nested PCR in combination with gold nanoparticle probe for hybridization demonsted high sensitivity and high specificity in identification of Mycobacterium tuberculosis complex (MTBC) and Mycobacterium tuberculosis (MTB) from clinical sputum samples. The sensitivity of this assay is dependent on whether there is enough DNA to be used. The detection steps are simple and rapid as long as enough target DNA is available.

Compared with conventional DNA detection systems whose specificity is mainly determined by the design of DNA primer sequences, the addition of specific gold nanoparticle probe in the invention further improves the specificity by reducing the background noise of non-specific DNA. The probes of the invention showed a sensitivity of 96.6% and specificity of 98.9% toward MTBC detection, and a sensitivity of 94.7% and specificity of 99.6% toward MTB detection were shown. In addition, the reading of the results can be conveniently achieved by direct observation besides spectrophotometric analysis if incuting time is long enough.

Meanwhile, the system of the invention can also be applied as a platform in DNA diagnosis other than MTBC and MTB detection. This method can be developed for automation analysis with spectrophotometric detection in large scale DNA diagnosis.

What is claimed is:

1. A probe for detecting Mycobacterium tuberculosis (MTB) and Mycobacterium tuberculosis complex (MTBC) in a specimen, wherein the probe is selected from the group consisting of nucleotide sequences of SEQ ID NO: 1 to SEQ ID NO: 4 and the completely complementary sequences thereof.

2. The probe as claimed in claim 1, wherein the nucleotide sequences of SEQ ID NO: 1 and the SEQ ID NO: 2 are complementarily hybridized to a fragment of IS6110 gene of Mycobacterium tuberculosis complex.

3. The probe as claimed in claim 2, wherein the fragment of IS6110 gene of Mycobacterium tuberculosis complex are amplified through primers consisting of nucleotide sequences of SEQ ID NO: 5 to SEQ ID NO: 8.

4. The probe as claimed in claim 1, wherein the nucleotide sequences of SEQ ID NO: 3 and SEQ ID NO: 4 are complementarily hybridized to a fragment of Rv3618 gene of Mycobacterium tuberculosis.

5. The probe as claimed in claim 4, wherein the fragment of Rv3618 gene of Mycobacterium tuberculosis is amplified through primers consisting of nucleotide sequences of SEQ ID NO: 9 to SEQ ID NO: 12.

6. The probe as claimed in claim 1, wherein the probe is labeled with a reporter selected from the group consisting of biotin, digoxigenin, fluorescence materials, nano-gold particles, nano-silver particles, nano-palladium (Pd), nano-ruthenium (Ru), nano-platinum (Pt) and any other conjugating color indicators.

7. A probe for detecting the presence of Mycobacterium tuberculosis complex, wherein the probe is selected from the group consisting of nucleotide sequences of SEQ ID NO: 1 to SEQ ID NO: 2 and the completely complementary sequences thereof.

8. The probe as claimed in claim 7, wherein the nucleotide sequences of SEQ ID NO: 1 and SEQ ID NO: 2 are complementarily hybridized to a fragment of IS6110 gene of Mycobacterium tuberculosis complex.

9. The probe as claimed in claim 8, wherein the fragment of IS6110 gene of Mycobacterium tuberculosis comple are amplified through primers consisting of nucleotide sequences of SEQ ID NO: 5 to SEQ ID NO: 8.

10. The probe as claimed in claim 7, wherein the probe is labeled with a reporter selected from the group consisting of biotin, digoxigenin, fluorescence materials, nano-gold particles, nano-silver particles, nano-palladium (Pd), nano-ruthenium (Ru), nano-platinum (Pt) and any other conjugating color indicators.

11. A probe for detecting the presence of Mycobacterium tuberculosis, wherein the probe is selected from the group consisting of nucleotide sequences of SEQ ID NO: 3 to SEQ ID NO: 4 and the completely complementary sequences thereof.

12. The probe as claimed in claim 11, wherein the nucleotide sequences of SEQ ID NO: 3 and SEQ ID NO: 4 are complementarily hybridized to a fragment of Rv3618 gene of Mycobacterium tuberculosis.

13. The probe as claimed in claim 12, wherein the fragment of Rv3618 gene of Mycobacterium tuberculosis are amplified through primers consisting of nucleotide sequences of SEQ ID NO: 9 to SEQ ID NO: 12.

14. The probe as claimed in claim 11, wherein the probe is labeled with a reporter elected from the group consisting of biotin, digoxigenin, fluorescence materials, nano-gold particles, nano-silver particles, nano-palladium (Pd), nano-ruthenium (Ru), nano-platinum (Pt) and any other conjugating color indicators.

15. A method for the identification of Mycobacterium tuberculosis and Mycobacterium tuberculosis complex, comprising:
(1) obtaining a specimen;
(2) hybridizing a DNA in the specimen with a first probe set having nucleotide sequences of SEQ ID NO: 1 and SEQ ID NO: 2, and a second probe set having nucleotide sequences of SEQ ID NO: 3 and SEQ ID NO: 4, or the completely complementary sequences thereof respectively; and
(3) determining the presence of Mycobacterium tuberculosis or Mycobacterium tuberculosis complex in a specimen.

16. The method as claimed in claim 15, wherein the nucleotide sequences of SEQ ID NO: 1 and SEQ ID NO. 2 are complementarily hybridized to a fragment of IS6110 gene of Mycobacterium tuberculosis complex.

17. The method as claimed in claim 16, wherein the fragment of IS6110 gene of Mycobacterium tuberculosis complex is an amplification product of nucleotide sequences of SEQ ID NO: 5 to SEQ ID NO: 8.

18. The method as claimed in claim 15, wherein the nucleotide sequences of SEQ ID NO: 3 and SEQ ID NO. 4 are complementarily hybridized to a fragment of Rv3618 gene of Mycobacterium tuberculosis.

19. The method as claimed in claim 18, wherein the fragment of Rv3618 gene of Mycobacterium tuberculosis is the amplification product of nucleotide sequences of SEQ ID NO: 9 to SEQ ID NO: 12.

20. The method as claimed in claim 15, wherein the probe is labeled with a reporter selected from the group consisting of biotin, digoxigenin, fluorescence materials, nano-gold particles, nano-silver particles, nano-palladium (Pd), nano-ruthenium (Ru), nano-platinum (Pt) and any other conjugating color indicators.


SEQUENCE LISTING (12 sequences)

SEQ ID NO: 1 - DNA, 19 bp, Artificial Sequence
  Probe for IS6110 gene (MTBC)
cacccatcgtctggagtgg

SEQ ID NO: 2 - DNA, 17 bp, Artificial Sequence
  Probe for IS6110 gene (MTBC)
ccaagcggatgcaccgg

SEQ ID NO: 3 - DNA, 19 bp, Artificial Sequence
  Probe for Rv3618 gene (MTB)
agccatgatttcgccatcg

SEQ ID NO: 4 - DNA, 17 bp, Artificial Sequence
  Probe for Rv3618 gene (MTB)
ctgggcacgtcgctgtt

SEQ ID NO: 5 - DNA, 20 bp, Artificial Sequence
  Primer for amplification of IS6110 (MTBC)
cgtgagggcatcgaggtggc

SEQ ID NO: 6 - DNA, 20 bp, Artificial Sequence
  Primer for amplification of IS6110 (MTBC)
gcgtaggcgtcggtgacaaa

SEQ ID NO: 7 - DNA, 20 bp, Artificial Sequence
  Primer for amplification of IS6110 (MTBC)
ctcgtccagcgccgcttcgg

SEQ ID NO: 8 - DNA, 20 bp, Artificial Sequence
  Primer for amplification of IS6110 (MTBC)
gcgtcggtgacaaaggccac

SEQ ID NO: 9 - DNA, 16 bp, Artificial Sequence
  Primer for amplification of Rv3618 (MTB)
attgcacatccgcccc

SEQ ID NO: 10 - DNA, 16 bp, Artificial Sequence
  Primer for amplification of Rv3618 (MTB)
ggacaaaccctgccgc

SEQ ID NO: 11 - DNA, 16 bp, Artificial Sequence
  Primer for amplification of Rv3618 (MTB)
cgactggttcaccctg

SEQ ID NO: 12 - DNA, 18 bp, Artificial Sequence
  Primer for amplification of Rv3618 (MTB)
taacagcgacgtgcccag